(19) United States
(12) Patent Application Publication
(10) Pub. No.: US 2020/0407355 A1
(43) Pub. Date: Dec. 31, 2020

(54) CRYSTALLINE FORMS OF VENETOCLAX

(71) Applicant: Olon S.P.A., Rodano (MI) (IT)

(72) Inventors: Barbara Novo, Rodano (MI) (IT); Jacopo Bonanomi, Rodano (MI) (IT); Gaia Migliazza, Rodano (MI) (IT)

(73) Assignee: Olon S.P.A., Rodano (MI) (IT)

(21) Appl. No.: 16/978,248

(22) PCT Filed: Feb. 28, 2019
(86) PCT No.: PCT/IB2019/051613

§ 371 Date: Sep. 4, 2020

(30) Foreign Application Priority Data
Mar. 5, 2018 (IT) 102018000003284

(51) Int. Cl. C07D 471/04

(52) U.S. Cl. CPC C07D 471/04; C07B 2200/13

(57) ABSTRACT

Disclosed are new crystalline forms of venetoclax, a selective Bcl2 inhibitor used as a chemotherapy agent, and the processes for preparation of said crystalline forms by treating venetoclax with suitable solvents.

### SUMMARY OF THE INVENTION

Disclosed are new crystalline forms of venetoclax and their preparation process.

### FIELD OF INVENTION

The present invention relates to new crystalline forms, called forms α, β, γ, δ, ε and θ, and the preparation thereof.

### BACKGROUND TO THE INVENTION

Apoptosis, also called “programmed cell death”, is not only an important biological phenomenon, but has also acquired enormous medical value; excessive apoptotic activity can cause disorders due to cell loss (such as Parkinson's disease), while a deficiency of apoptosis can involve uncontrolled cell growth, the underlying mechanism of tumours. Various studies have confirmed that proto-oncogene Blc-2 is the main substance involved in the apoptosis inhibition mechanism by means of expression of the Bcl-2 protein, overexpression of which is associated with resistance to chemotherapy in some lymphomas.

Venetoclax is a powerful, selective oral Blc-2 inhibitor, which was approved by the FDA on 11 Apr. 2016 under the tradename of Venclexta for the treatment of adult patients suffering from chronic lymphocytic leukaemia (CLL) who are unsuitable for or have undergone failed treatment with a B-cell receptor pathway inhibitor, even in the presence of the 17p deletion or the TP53 mutation.

Fourteen crystalline forms of venetoclax, called forms A, B, C, D, E, F, G, H, I, J, K, L, M and N are known in the literature and described in WO2012/071336A1. Their XRPD pattern is described as follows:

Crystalline form A presents an XRPD pattern containing the most intense peaks at 2θ=6.3, 7.1, 9.0, 9.5, 12.5, 14.5, 14.7, 15.9, 16.9, 18.9.

Crystalline form B presents an XRPD pattern containing the most intense peaks at 2θ=5.8, 7.7, 8.3, 9.9, 13.0, 13.3, 14.2, 15.3, 16.6, 17.9, 18.3, 19.8, 20.7, 21.2, 21.9, 22.5, 23.6, 24.1.

Crystalline form C presents an XRPD pattern containing the most intense peaks at 2θ=5.8, 7.6, 7.9, 10.7, 11.7, 14.0, 15.3, 15.8, 17.4, 18.3, 19.9, 20.4, 20.7, 22.5, 24.9, 25.8, 26.7.

Crystalline form D presents an XRPD pattern containing the most intense peaks at 2θ=3.3, 6.4, 7.1, 7.3, 10.1, 11.4, 13.2, 14.4, 14.6, 15.1, 15.8, 16.2, 17.2, 17.6, 18.0, 18.6, 19.0, 19.5, 19.8, 20.2, 20.7, 21.0, 22.5, 23.0, 26.0, 28.9, 29.2.

Crystalline form E presents an XRPD pattern containing the most intense peaks at 2θ=5.9, 7.1, 9.6, 10.0, 10.7, 11.1, 13.2, 14.8, 18.2.

Crystalline form F presents an XRPD pattern containing the most intense peaks at 2θ=5.8, 7.1, 9.5, 9.9, 10.6, 11.6, 13.1, 13.8, 14.8, 16.0, 17.9, 20.2, 21.2, 23.2, 24.4, 26.4.

Crystalline form G presents an XRPD pattern containing the most intense peaks at 2θ=3.3, 6.5, 7.0, 7.3, 9.2, 9.7, 11.2, 11.4, 11.9, 12.9, 14.4, 14.9, 15.8, 16.2, 17.2, 17.4, 17.8, 18.5, 18.9, 19.4, 20.1, 20.7, 20.9, 22.0, 22.7, 23.4, 23.8, 24.7, 25.9, 27.0, 28.9.

Crystalline form H presents an XRPD pattern containing the most intense peaks at 2θ=5.8, 7.4, 7.6, 10.2, 13.0, 13.6, 14.9, 16.4, 17.0, 17.5, 18.2, 19.4, 19.7, 20.4, 21.0, 21.2, 21.8, 22.4, 22.9, 24.2, 24.3, 26.1, 29.2.

Crystalline form I presents an XRPD pattern containing the most intense peaks at 2θ=6.4, 6.9, 7.7, 8.8, 9.4, 11.1, 12.3, 12.8, 16.5, 17.0, 17.4, 18.3, 18.6, 19.0, 19.2, 20.3, 21.6, 22.3, 22.9, 23.7.

Crystalline form J presents an XRPD pattern containing the most intense peaks at 2θ=6.0, 6.8, 8.0, 9.0, 9.7, 11.2, 11.9, 12.6, 14.7, 15.0, 15.2, 15.8, 16.4, 16.6, 17.6, 17.8, 17.9, 18.7, 20.2, 20.8, 21.6, 22.2, 22.6, 23.3, 23.8, 24.0, 24.4, 26.8, 27.1, 28.0, 28.2.

Crystalline form K presents an XRPD pattern containing the most intense peaks at 2θ=5.1, 5.9, 7.7, 9.9, 10.2, 10.8, 13.6, 14.0, 15.4, 15.9, 16.2, 17.6, 18.3, 18.7, 19.7, 19.9, 20.1, 20.4, 20.7, 20.9, 22.9, 26.2.

Crystalline form L presents an XRPD pattern containing the most intense peaks at 2θ=4.6, 8.7, 9.6, 9.9, 12.3, 14.9, 15.7, 17.6, 18.1, 18.4, 19.3, 19.6, 21.0, 23.3, 23.9, 24.8, 26.5, 27.2, 27.4, 29.0, 30.1.

Crystalline form M presents an XRPD pattern containing the most intense peaks at 2θ=4.8, 7.7, 8.3, 9.7, 10.2, 12.0, 12.6, 14.5, 15.4, 17.4, 17.9, 18.4, 19.1, 19.5, 21.0, 22.4, 23.3, 23.9, 25.1, 26.8.

Crystalline form N presents an XRPD pattern containing the most intense peaks at 2θ=4.0, 4.6, 8.0, 8.5, 9.4, 14.6, 17.1, 17.4, 17.8, 18.1, 19.2, 19.5, 20.1, 20.4, 20.5, 21.7.

Four other crystalline forms of venetoclax, called forms b, d, f, and g, are known and described in WO2017/063572A1. Their XRPD pattern is described as follows:

Crystalline form b presents an XRPD pattern containing the most intense peaks at 2θ=5.3, 5.9, 6.7, 10.2, 11.3, 15.7, 16.8, 20.6, 22.8.

Crystalline form d presents an XRPD pattern containing the most intense peaks at 2θ=6.3, 11.4, 12.7, 16.4, 16.8, 19.1, 19.9, 22.3, 22.9.

Crystalline form f presents an XRPD pattern containing the most intense peaks at 2θ=5.9, 12.4, 13.3, 17.5, 17.9, 18.5, 19.0, 21.3, 24.2.

Crystalline form g presents an XRPD pattern containing the most intense peaks at 2θ=9.6, 10.6, 11.1, 11.8, 14.6, 16.5, 19.3, 20.3, 24.5.

### DESCRIPTION OF FIGURES

FIG. 1: Infrared spectrum of venetoclax crystalline form α.

FIG. 2: DSC curve of venetoclax crystalline form α.

FIG. 3: XRPD pattern of venetoclax crystalline form α.

FIG. 4: ¹H-NMR spectrum in d₆-DMSO of venetoclax crystalline form α.

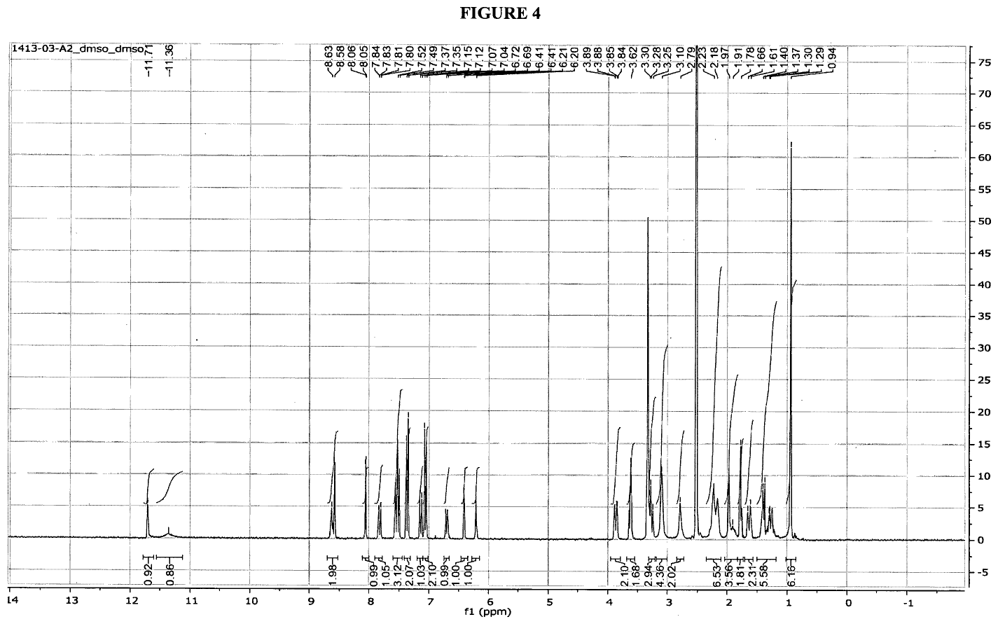

FIG. 5: Infrared spectrum of venetoclax crystalline form β.

FIG. 6: DSC curve of venetoclax crystalline form β.

FIG. 7: XRPD pattern of venetoclax crystalline form β.

FIG. 8: ¹H-NMR spectrum in d₆-DMSO of venetoclax crystalline form β.

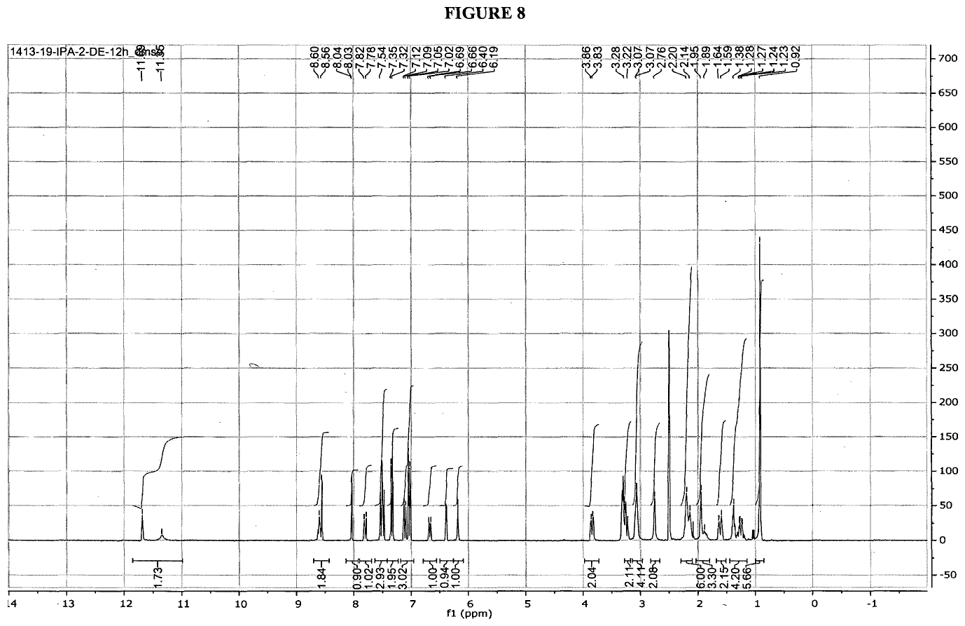

FIG. 9: Infrared spectrum of venetoclax crystalline form γ.

FIG. 10: DSC curve of venetoclax crystalline form γ.

FIG. 11: XRPD pattern of venetoclax crystalline form γ.

FIG. 12: ¹H-NMR spectrum in d₆-DMSO of venetoclax crystalline form γ.

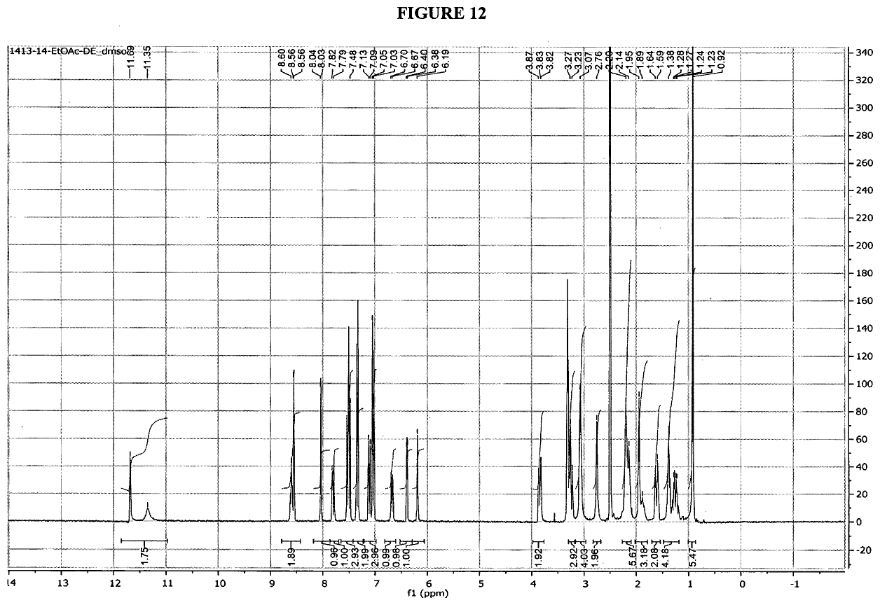

FIG. 13: Infrared spectrum of venetoclax crystalline form δ.

FIG. 14: DSC curve of venetoclax crystalline form δ.

FIG. 15: XRPD pattern of venetoclax crystalline form δ.

FIG. 16: ¹H-NMR spectrum in d₆-DMSO of venetoclax crystalline form δ.

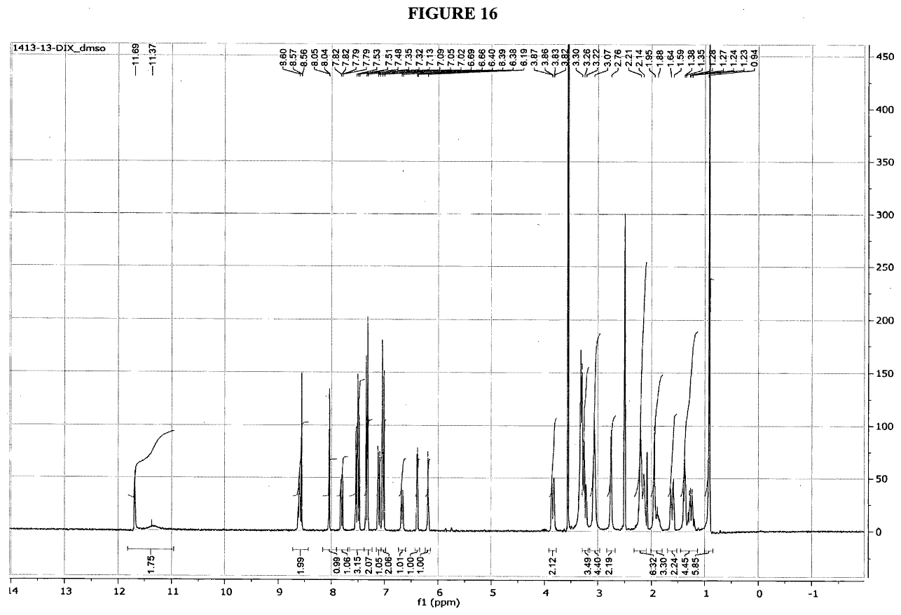

FIG. 17: Infrared spectrum of venetoclax crystalline form ε.

FIG. 18: DSC curve of venetoclax crystalline form ε.

FIG. 19: XRPD pattern of venetoclax crystalline form ε.

FIG. 20: ¹H-NMR spectrum in d₆-DMSO of venetoclax crystalline form ε.

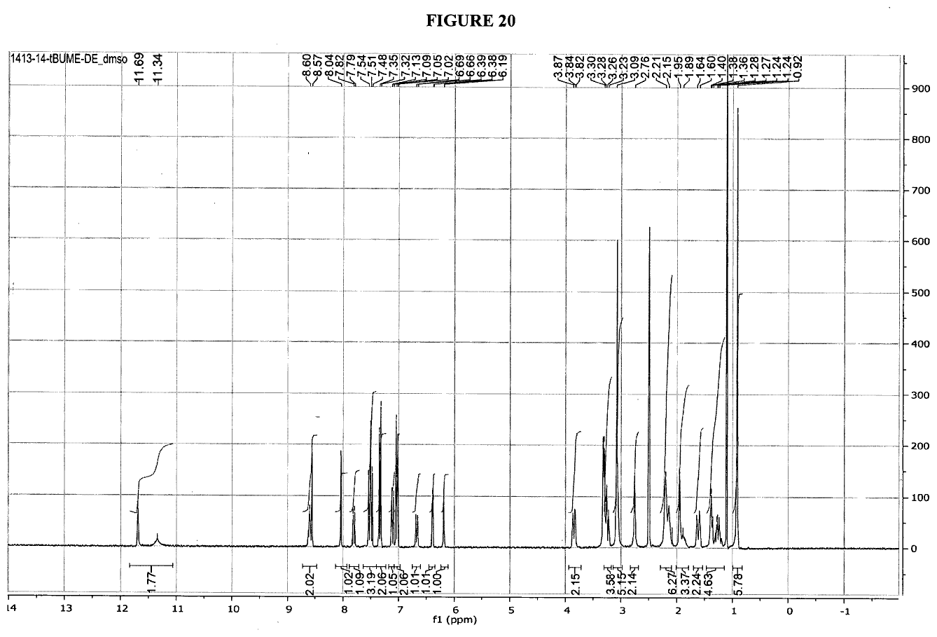

FIG. 21: Infrared spectrum of venetoclax crystalline form θ.

FIG. 22: DSC curve of venetoclax crystalline form θ.

FIG. 23: XRPD pattern of venetoclax crystalline form θ.

FIG. 24: ¹H-NMR spectrum in d₆-DMSO of venetoclax crystalline form θ.

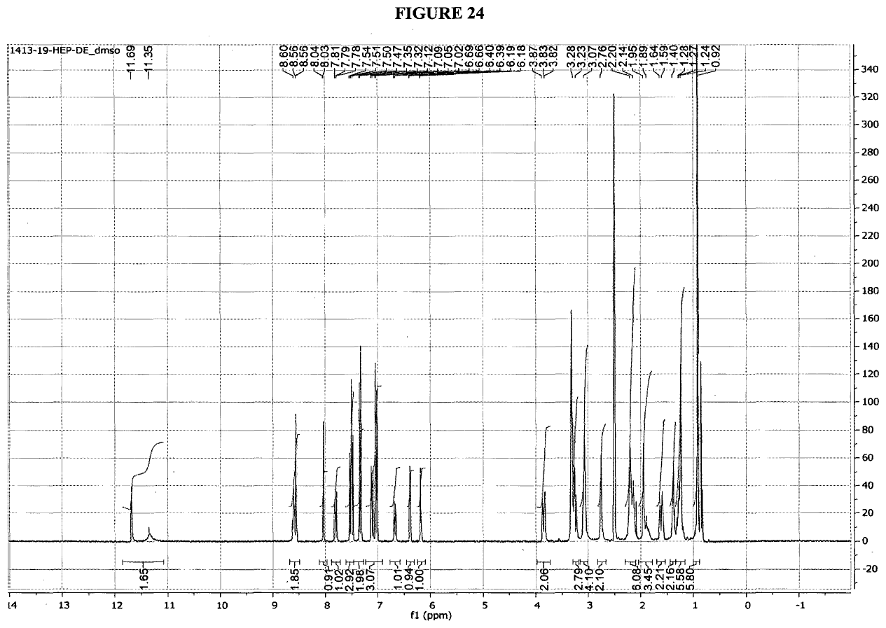

### DESCRIPTION OF THE INVENTION

The present invention relates to the preparation and characterisation of crystalline forms α, β, γ, δ, ε and θ of venetoclax.

The manufacture of an amorphous compound can sometimes be inconvenient, because the physical properties of the compound can adversely affect the manufacturing process. In particular, an amorphous compound cannot be purified by crystallisation, and it is therefore very difficult to obtain a highly pure finished product without using onerous purification techniques such as chromatography. An amorphous compound can also become physically unstable during manufacture at the formulation stage, and may therefore give rise to unexpected phase transitions and crystallise into undesirable polymorphs.

However, the crystalline forms can be used to modulate and/or improve the physicochemical characteristics of the API, regulating properties relating to the solid state (hygroscopicity, melting point, etc.), pharmaceutical formulations (degree of solubility/dissolution, stability, etc.) and crystallisation characteristics (purity, yield, etc).

Molecules able to induce apoptosis are particularly dependent on said properties, which influence the manufacture, formulation, storage and transport of the API. Crystalline form α can be obtained by crystallisation from a solution of venetoclax in a polar aprotic solvent such as tetrahydrofuran. This process gives rise to a crystal with high purity, and thus offers great advantages, primarily precise control of the process, which directly produces a product substantially devoid of impurities, and secondly a reduction in the process costs which may be required to eliminate impurities deriving from synthesis.

Conversely, forms β, γ, δ, ε and θ can be obtained by treating venetoclax in a suspension of various solvents, including isopropanol or ethyl acetate which, being characterised by low toxicity, enable the maximum recommended exposure limit to be maintained at a higher level than those of other solvents generally used in the pharmaceutical industry. All this can offer various advantages, such as elimination of impurities from the finished product by means of treatment in cheap, low-toxicity, environment-friendly solvents.

Preparation of Venetoclax Crystalline Form α:

Venetoclax crystalline form α can be obtained by crystallisation, for example after suspension of any form of venetoclax (amorphous form, or crystalline form A, B, C, D, E, F, G, H, I, J, N, b, d, f or g) in a suitable polar aprotic solvent such as tetrahydrofuran, at room temperature. The suspension is heated, until completely dissolved, at a temperature ranging between 16° C. and 65° C., preferably between 20° C. and 60° C., and more preferably between 30° C. and 55° C. The resulting solution is then left under stirring for between 1 and 48 hours, preferably between 10 and 24 hours, and more preferably between 2 and 12 hours, and cooled to a temperature ranging between 10° C. and 37° C., preferably between 15° C. and 35° C., and more preferably between 20° C. and 30° C., until a suspension is again obtained. The resulting crystal is then recovered by filtration and dried under vacuum.

Alternatively, venetoclax crystalline form α can be prepared by thin-layer evaporation by dissolving any form of venetoclax (amorphous form or crystalline form A, B, C, D, E, F, G, H, I, J, N, b, d, f or g) in a suitable polar aprotic solvent such as tetrahydrofuran, at a temperature ranging between 16° C. and 60° C., preferably between 20° C. and 50° C., and more preferably between 25° C. and 45° C. The resulting solution is then left under stirring for a time ranging between 1 and 12 hours, preferably between 1 and 6 hours, and more preferably between 1 and 2 hours, and cooled to a temperature ranging between 10° C. and 50° C., preferably between 15° C. and 40° C., and more preferably between 20° C. and 30° C. The solution is filtered through an 0.45 μm Whatman filter and left to evaporate at a temperature ranging from 0° C. to 60° C., preferably from 10° C. to 45° C., and even more preferably from 20° C. to 30° C., and at atmospheric pressure.

Alternatively, venetoclax crystalline form α can be obtained by precipitation with anti-solvent, for example after complete dissolution of any form of venetoclax (amorphous form or crystalline form A, B, C, D, E, F, G, H, I, J, N, b, d, f or g) in a suitable polar aprotic solvent such as tetrahydrofuran. The resulting solution is left under stirring for a time ranging between 1 and 12 hours, preferably between 1 and 6 hours, and more preferably between 1 and 2 hours, at a temperature ranging between 10° C. and 50° C., preferably between 15° C. and 40° C., and more preferably between 20° C. and 30° C. A polar anti-solvent such as water, methanol, ethanol, 1-butanol, 1-propanol, isopropanol, methyl ethyl ketone, acetone, ethyl acetate, dioxane, acetonitrile, isopropyl acetate, isobutyl acetate, dichloromethane, methyltetrahydrofuran, isopropyl ether, tert-butyl methyl ether, cyclopentyl methyl ether, toluene, cyclohexane or heptane, more preferably water, is then added to the solution to precipitate the crystal, which is recovered by filtration and dried under vacuum. Alternatively, the solution of venetoclax crystalline form α dissolved in a suitable polar aprotic solvent such as tetrahydrofuran is added to a polar anti-solvent such as water, methanol, ethanol, 1-butanol, 1-propanol, isopropanol, methyl ethyl ketone, acetone, ethyl acetate, dioxane, acetonitrile, isopropyl acetate, isobutyl acetate, dichloromethane, methyltetrahydrofuran, isopropyl ether, tert-butyl methyl ether, cyclopentyl methyl ether, toluene, cyclohexane or heptane, more preferably water, to precipitate the crystal, which is recovered by filtration and dried under vacuum.

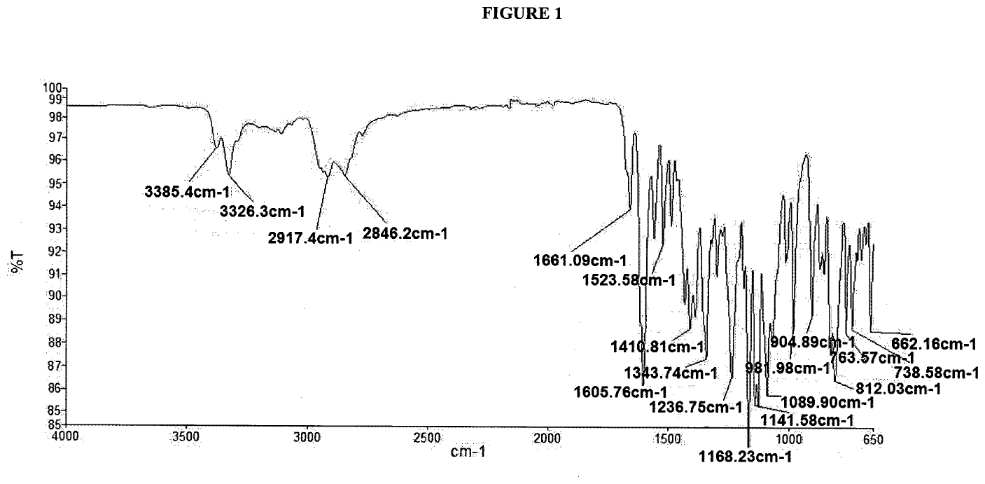

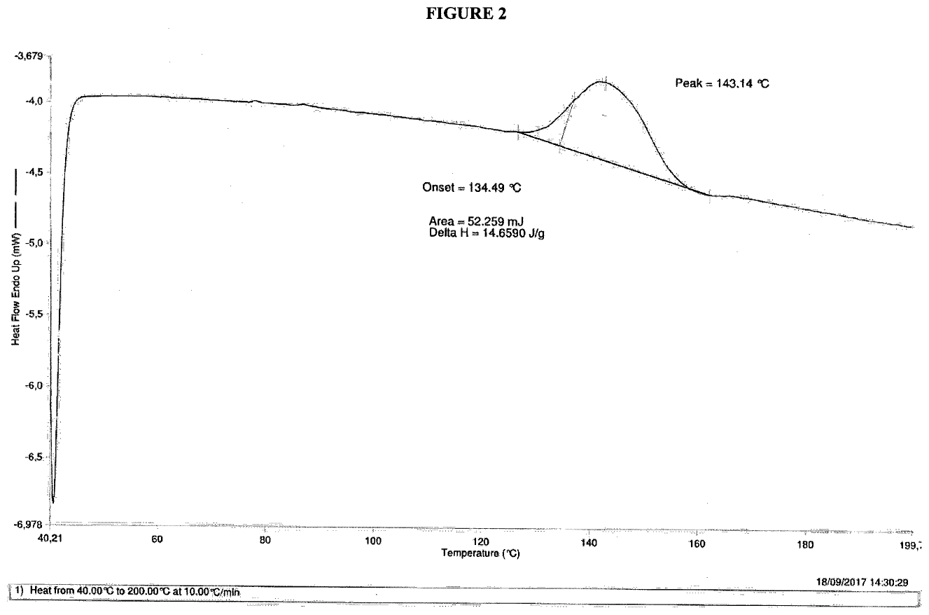

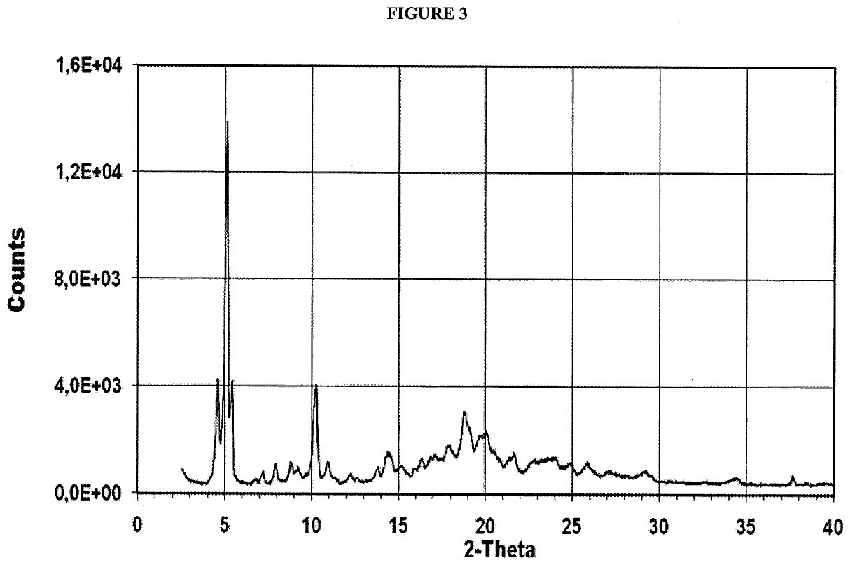

Venetoclax crystalline form α presents an IR spectrum, DSC curve and XRPD pattern as shown in FIGS. 1, 2 and 3 respectively.

In particular, crystalline form α of venetoclax presents:
An IR spectrum comprising absorption peaks at 3385.4, 3326.3, 2917.4, 2846.2, 1161.1, 1605.8, 1523.6, 1410.8, 1343.7, 1236.8, 1168.2, 1141.6, 1089.9, 981.9, 904.9, 812.1, 763.6, 738.6, 662.2±1.5 cm⁻¹;
A DSC pattern comprising an endothermic peak at 142±1° C.;
An XRPD pattern obtained at the CuKα wavelength comprising the following peaks: (2θ): 4.61, 5.14, 5.44, 7.22, 7.98, 8.80, 9.21, 10.27, 10.95, 12.27, 13.84, 14.36, 15.15, 16.35, 17.96, 18.77, 20.02, 21.62, 24.91, 25.86, 29.19±0.2°.

Preparation of Venetoclax Crystalline Forms β, γ, δ, ε and θ:

Crystalline forms β, γ, δ, ε and θ of venetoclax can be obtained by the same process of suspension of any crystalline form of venetoclax (amorphous form or crystalline form A, B, C, D, E, F, G, H, I, J, N, b, d, f, g or a) in suitable solvents such as isopropanol, n-propanol, ethanol, n-butanol, t-butanol or methanol, and more preferably isopropanol for form β; ethyl acetate, isopropyl acetate and isobutyl acetate, dichloromethane or methyltetrahydrofuran, and more preferably ethyl acetate, for form γ; dioxane for form δ; methyl tert-butyl ether for form ε; heptane, cyclohexane, n-hexane or pentane, more preferably heptane, for form θ. The suspension is maintained under stirring for a time ranging between 1 and 48 hours, preferably between 12 and 36 hours, and more preferably between 18 and 28 hours, at a temperature ranging between 10° C. and 37° C., preferably between 15° C. and 35° C., and more preferably between 20° C. and 30° C. The crystal is then recovered by filtration and dried under vacuum.

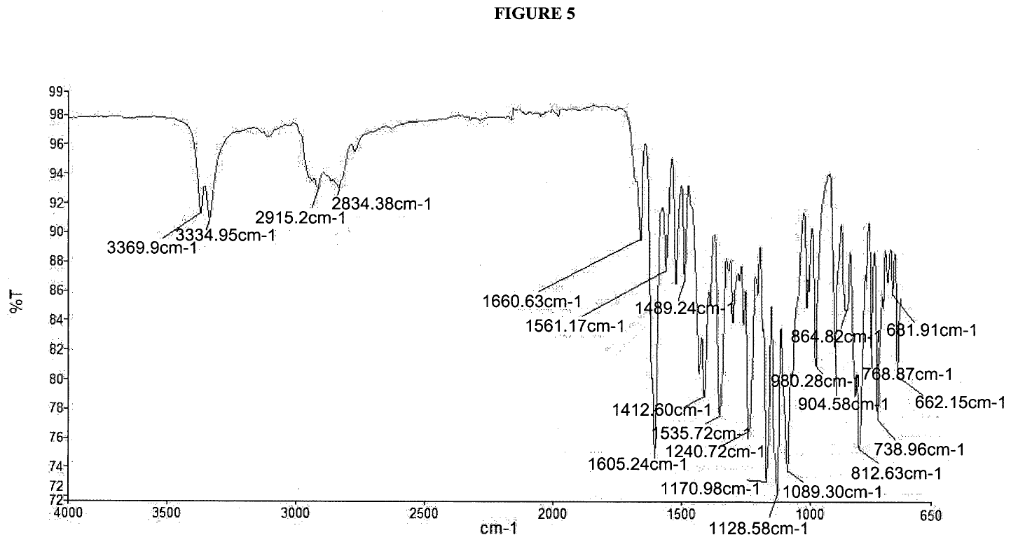

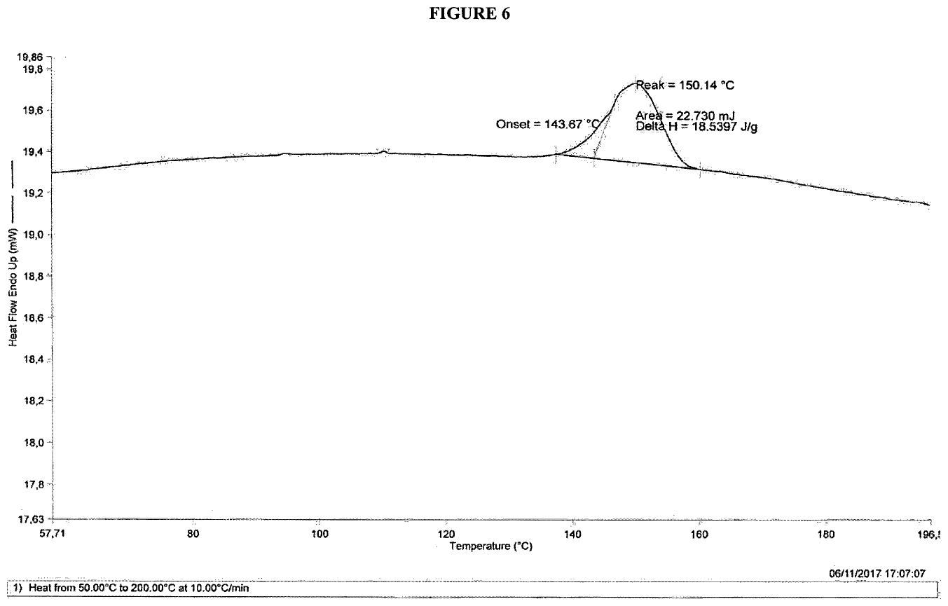

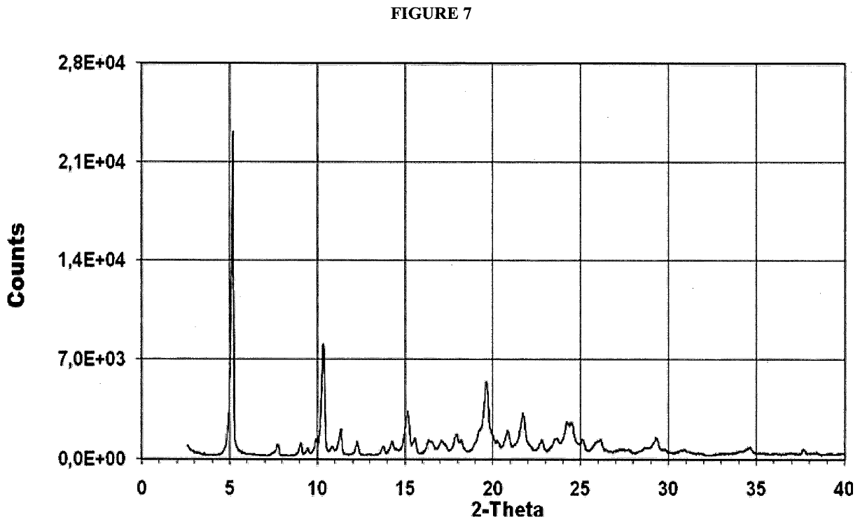

Venetoclax crystalline form β presents an IR spectrum, DSC curve and XRPD pattern as shown in FIGS. 5, 6 and 7 respectively.

In particular, the crystalline form β of venetoclax presents:
An IR spectrum comprising absorption peaks at 3369.9, 3335, 2915.2, 2834.4, 1160.6, 1605.2, 1561.2, 1489.2, 1412.6, 1353.7, 1240.7, 1171, 1128.6, 1089.3, 980.3, 904.6, 864.8, 812.6, 768.9, 739, 681.9, 662.2±1.5 cm⁻¹;
A DSC pattern comprising an endothermic peak at 150.14±1° C.;
An XRPD pattern at the wavelength CuKα comprising the following peaks (2θ): 5.2, 7.77, 9.07, 9.46, 9.96, 10.35, 10.83, 11.34, 12.28, 13.75, 14.28, 15.16, 15.58, 16.35, 17.08, 17.95, 18.22, 19.62, 20.86, 21.75, 22.78, 23.65, 24.20, 24.44, 25.07, 26.03, 29.27±0.2°.

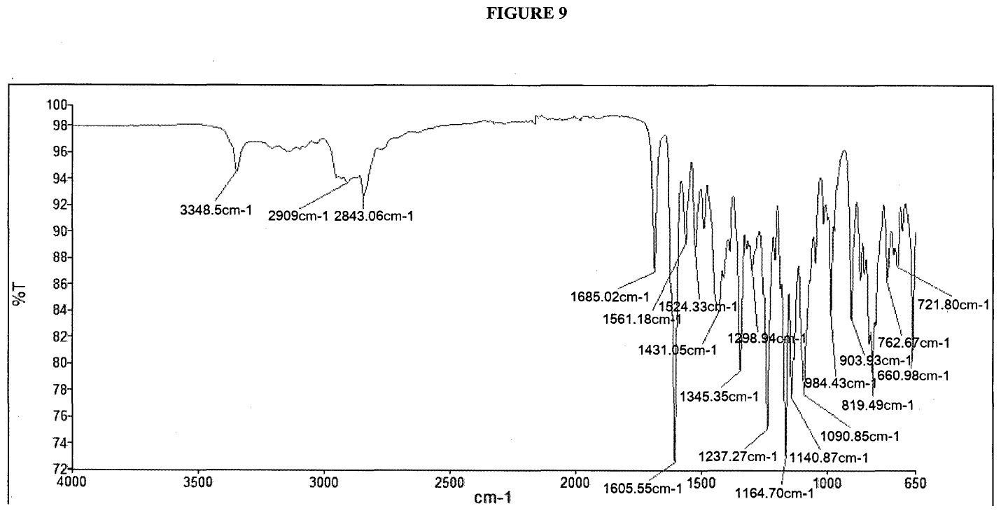

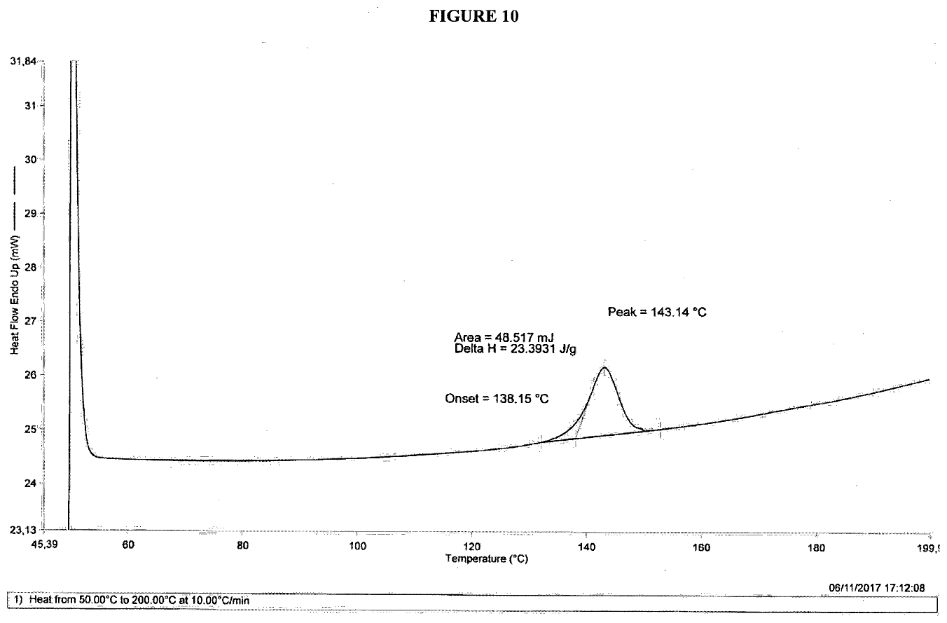

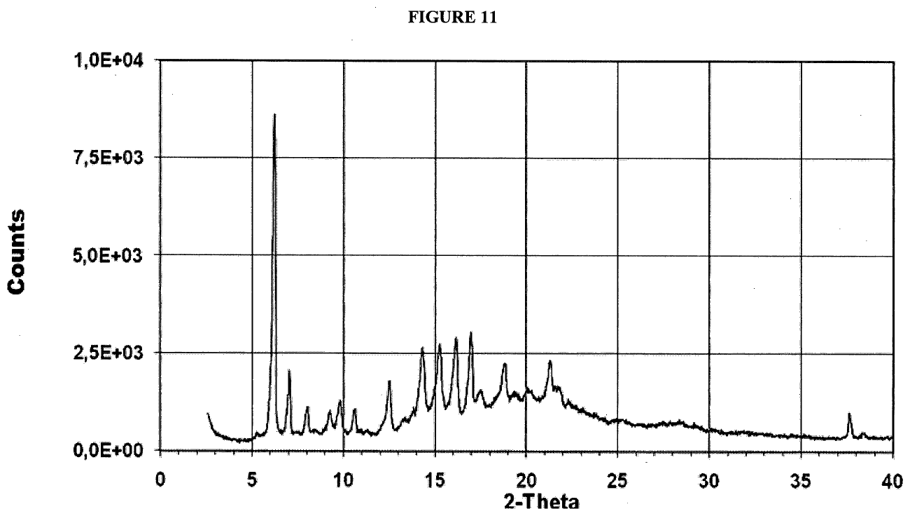

Venetoclax crystalline form γ presents an IR spectrum, DSC curve and XRPD pattern as shown in FIGS. 9, 10 and 11 respectively.

In particular, the crystalline form γ of venetoclax presents:
An IR spectrum comprising absorption peaks at 3348.5, 2909, 2843.1, 1685, 1605.7, 1561.2, 1524.3, 1431.1, 1345.4, 1298.9, 1237.3, 1164.7, 1140.9, 1090.9, 984.4, 903.9, 819.5, 762.7, 721.8, 661±1.5 cm⁻¹;
A DSC pattern comprising an endothermic peak at 143.14±1° C.;
An XRPD pattern at the CuKα wavelength comprising the following peaks (2θ): 6.23, 7.04, 8.02, 9.27, 9.82, 10.65, 12.52, 14.32, 15.26, 16.17, 16.98, 17.49, 18.84, 19.32, 20.04, 21.32±0.2°.

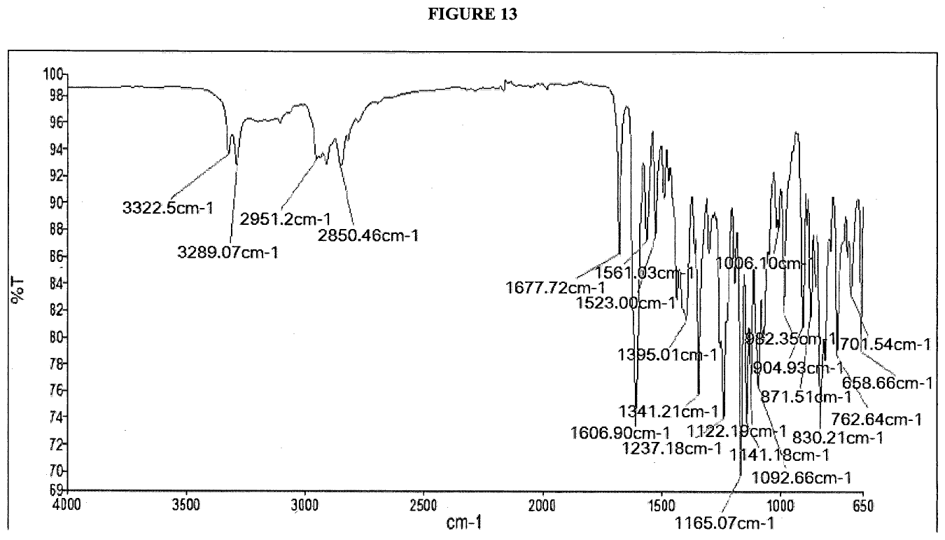

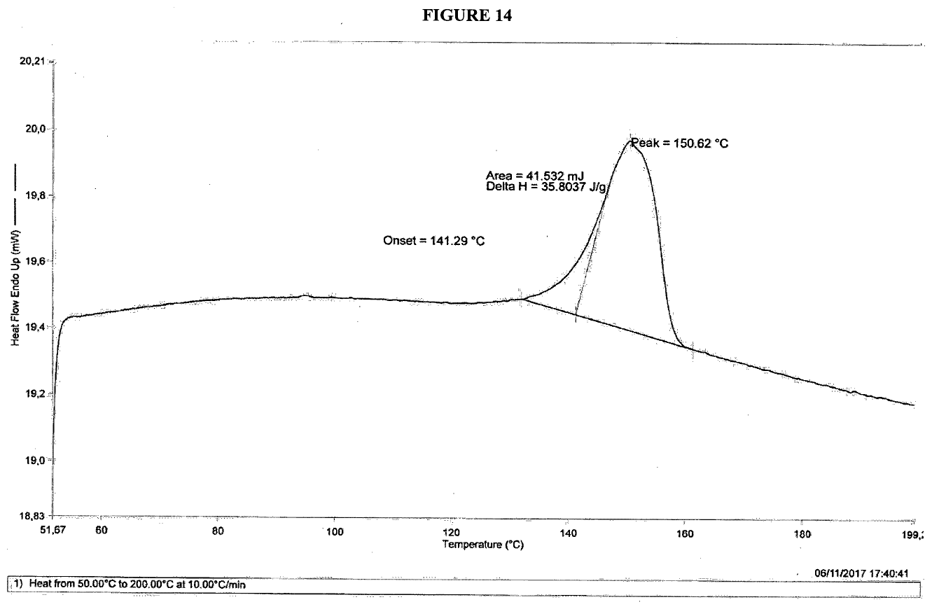

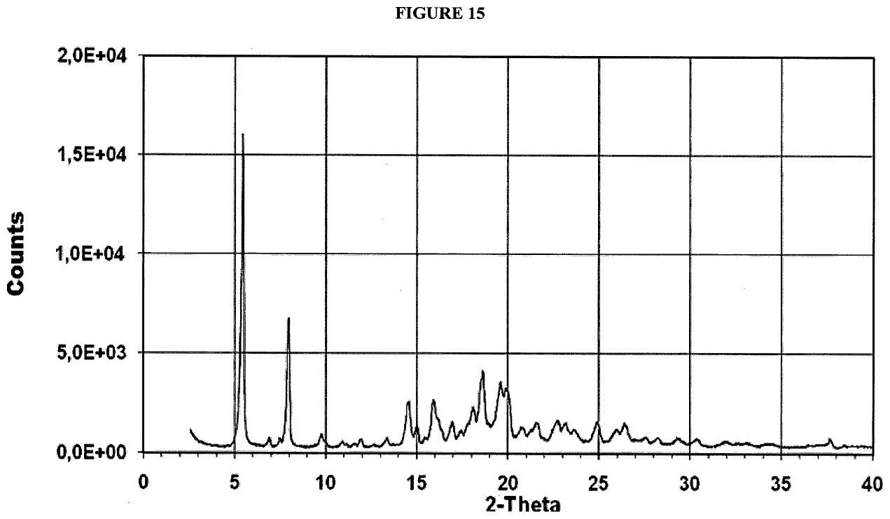

Venetoclax crystalline form δ presents an IR spectrum, DSC curve and XRPD pattern as shown in FIGS. 13, 14 and 15 respectively.

In particular, crystalline form δ of venetoclax presents:
An IR spectrum comprising absorption peaks at 3322.5, 3289.1, 2951.2, 2850.5, 1677.7, 1606.9, 1561, 1523, 1395, 1341.2, 1237.2, 1165.1, 1122.2, 1141.2, 1092.7, 1006.1, 982.4, 904.9, 871.5, 830.2, 762.6, 701.5, 658.7±1.5 cm⁻¹;
A DSC pattern comprising an endothermic peak at 150.62±1° C.;
An XRPD pattern at the CuKα wavelength comprising the following peaks (2θ): 5.46, 6.92, 7.63, 7.98, 9.78, 10.96, 11.59, 11.95, 13.39, 14.55, 15.01, 15.46, 15.91, 16.96, 17.47, 18.14, 18.62, 19.6, 19.92, 20.73, 21.54, 22.72, 23.19, 23.59, 24.91, 25.93, 26.39, 27.59, 28.26, 29.27, 30.33±0.2°.

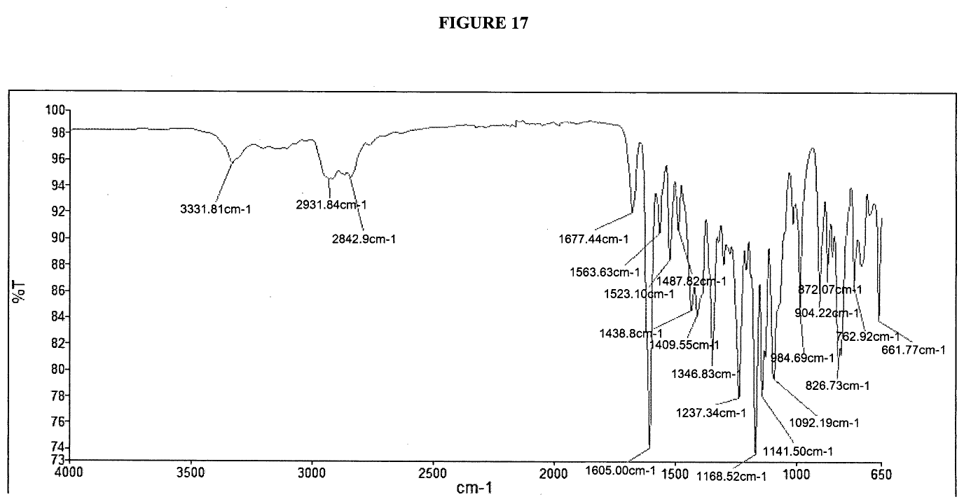

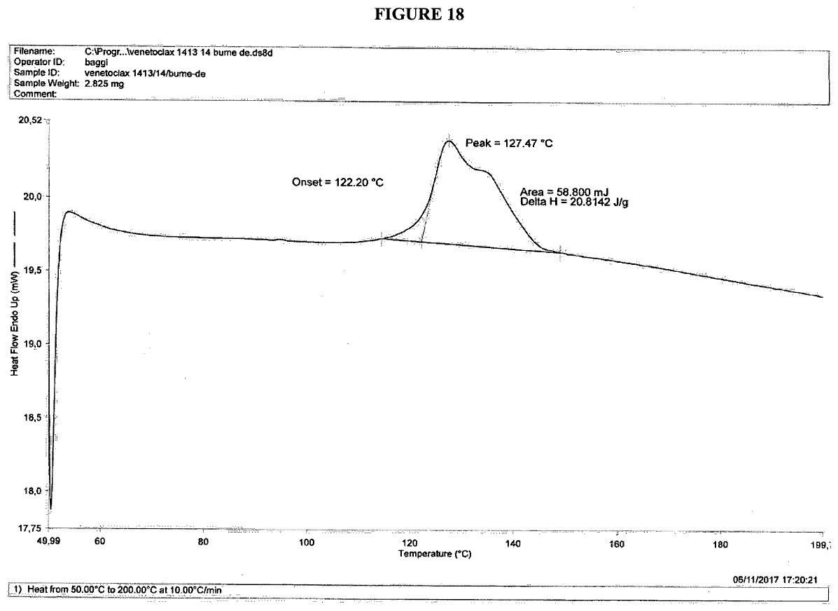

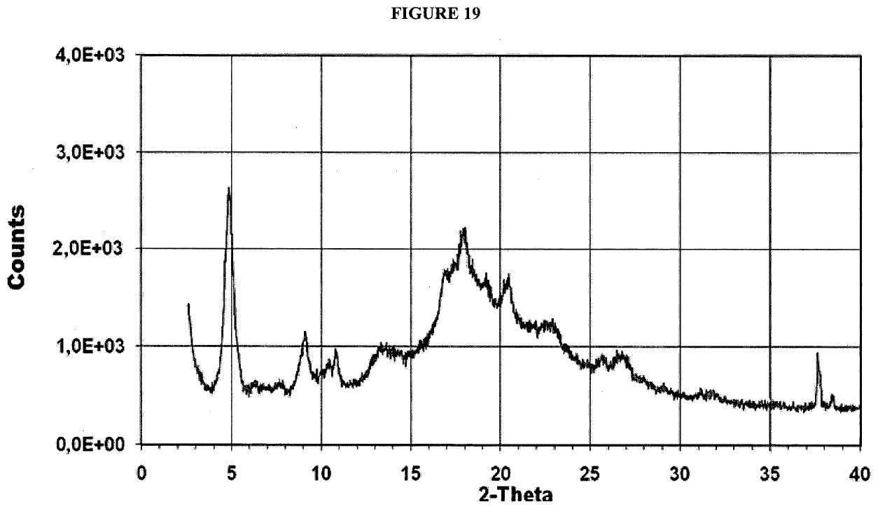

Venetoclax crystalline form ε presents an IR spectrum, DSC curve and XRPD pattern as shown in FIGS. 17, 18 and 19 respectively.

In particular, crystalline form ε of venetoclax presents:
An IR spectrum comprising absorption peaks at 3331.8, 2931.8, 2842.9, 1677.4, 1605, 1563.6, 1523.1, 1487.8, 1433.8, 1409.6, 1346.8, 1237.3, 1168.5, 1141.5, 1092.2, 984.7, 904.2, 872.1, 826.7, 762.9, 661.8±1.5 cm⁻¹;
A DSC pattern comprising an endothermic peak at 127.47±1° C.;
An XRPD pattern at the wavelength CuKα comprising the following peaks (2θ): 4.85, 9.11, 10.8, 13.35, 16.98, 17.04, 18.06, 19.11, 20.45, 23.8, 25.64, 26.47±0.2°.

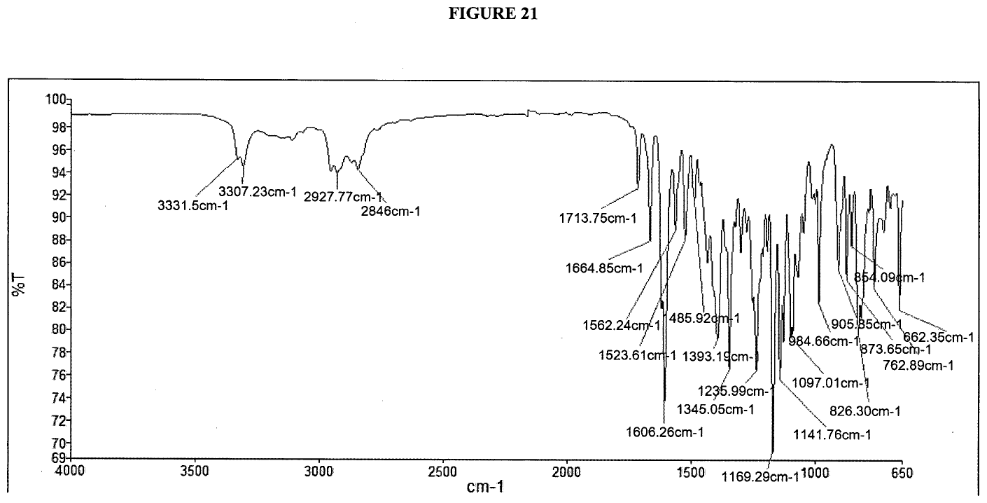

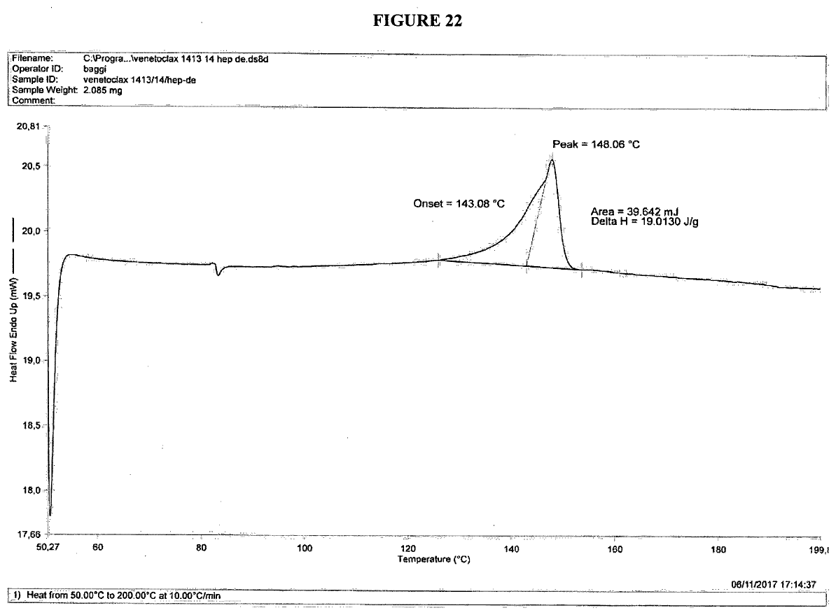

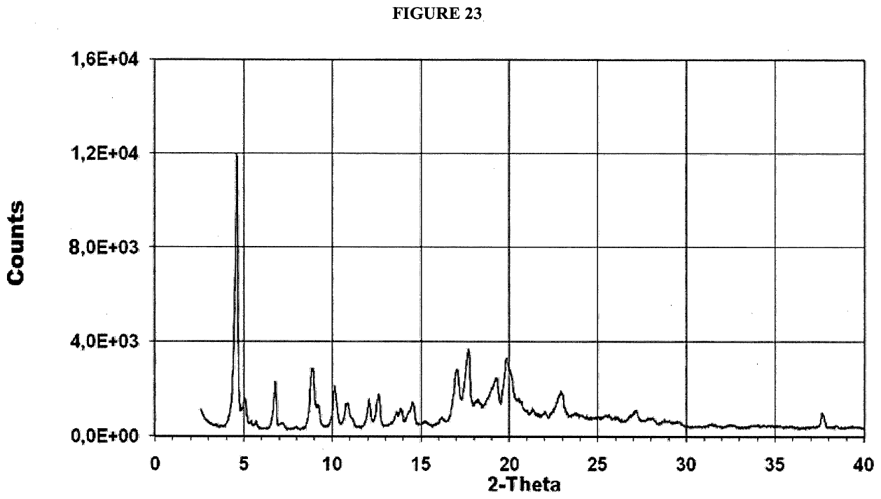

Venetoclax crystalline form θ presents an IR spectrum, DSC curve and XRPD pattern as shown in FIGS. 21, 22 and 23 respectively.

In particular, the crystalline form θ of venetoclax presents:
An IR spectrum comprising absorption peaks at 3331.5, 3307.2, 2927.8, 2846, 1713.8, 1664.9, 1562.2, 1523.6, 1606.3, 1485.9, 1393.2, 1345.1, 1236, 1169.3, 1141.8, 1097, 984.7, 905.9, 873.7, 854.1, 826.3, 762.9, 662.4±1.5 cm⁻¹;
A DSC pattern comprising an endothermic peak at 148.06±1° C.;
An XRPD pattern at the wavelength CuKα comprising the following peaks (2θ): 4.61, 5.10, 5.44, 5.72, 6.80, 8.84, 9.21, 10.15, 10.90, 12.09, 12.64, 13.89, 14.53, 15.28, 16.19, 17.06, 17.69, 18.22, 19.25, 19.98, 21.32, 22.03, 22.90, 27.10±0.2°.

### EXAMPLES

The IR spectra were recorded with a Perkin Elmer Frontier FT-RL instrument with universal ATR sampling accessory. The spectrum is recorded by performing 16 scans at a resolution of 4 cm⁻¹.

The DSC patterns were recorded with a Perkin Elmer Pyrisl instrument, and 3-5 mg of material were used to prepare the samples. The scans were conducted at the rate of 10° C. a minute.

The NMR spectra were recorded with a Varian Mercury 300 instrument in DMSO at 25° C., 16 scans being performed.

The XRPD spectra were recorded with a Bruker D2 instrument which uses the following parameters: Wavelength CuKα (λ=15419 A)—Energy 30 KV—Stepsize: 0.02°—2θ Range: 2.6°-40°.

### Example 1

Venetoclax free base (100 mg) is dissolved in 4 ml of tetrahydrofuran and the solution is stirred for about an hour, then filtered through an 0.45 μm Whatman filter, transferred to a watch glass and left to evaporate at a temperature of 25° C. and atmospheric pressure for at least 12 hours. The product (crystalline form α) presents an IR spectrum, DSC curve and XRPD pattern as shown in FIGS. 1-3 respectively.

### Example 2

Venetoclax free base (750 mg) is suspended in 10 ml of tetrahydrofuran under magnetic stirring at ambient temperature and pressure. The suspension is then dissolved at a temperature ranging between 50 and 55° C. under magnetic stirring until completely dissolved. The solution is cooled to 25° C. and left under stirring for at least 12 hours until a suspension is again obtained. The resulting crystal is isolated by filtration. The product (crystalline form α) presents an IR spectrum, DSC curve and XRPD pattern as shown in FIGS. 1-3 respectively.

### Example 3

Venetoclax free base (100 mg) is dissolved in 2 ml of tetrahydrofuran under magnetic stirring for about an hour, then filtered through an 0.45 μm Whatman filter. 3 ml of water at ambient temperature is added rapidly to the resulting solution to precipitate the crystal, which is recovered by filtration and dried under vacuum. The product (crystalline form α) presents an IR spectrum, DSC curve and XRPD pattern as shown in FIGS. 1-3 respectively.

### Example 4

Venetoclax free base (100 mg) is dissolved in 2 ml of tetrahydrofuran under magnetic stirring for about an hour, then filtered through an 0.45 μm Whatman filter. 3 ml of water at the temperature of 0-5° C. is added rapidly to the resulting solution to precipitate the crystal, which is recovered by filtration and dried under vacuum. The product (crystalline form α) presents an IR spectrum, DSC curve and XRPD pattern as shown in FIGS. 1-3 respectively.

### Example 5

Venetoclax free base (100 mg) is dissolved in 2 ml of tetrahydrofuran under magnetic stirring for about an hour, then filtered through an 0.45 μm Whatman filter. 3 ml of water at ambient temperature is added drop by drop to the resulting solution to precipitate the crystal, which is recovered by filtration and dried under vacuum. The product (crystalline form α) presents an IR spectrum, DSC curve and XRPD pattern as shown in FIGS. 1-3 respectively.

### Example 6

Venetoclax free base (100 mg) is dissolved in 2 ml of tetrahydrofuran under magnetic stirring for about an hour, then filtered through an 0.45 μm Whatman filter. 3 ml of water at the temperature of 0-5° C. is added drop by drop to the resulting solution to precipitate the crystal, which is recovered by filtration and dried under vacuum. The product (crystalline form α) presents an IR spectrum, DSC curve and XRPD pattern as shown in FIGS. 1-3 respectively.

### Example 7

Venetoclax free base (100 mg) is dissolved in 2 ml of tetrahydrofuran under magnetic stirring for about an hour, then filtered through an 0.45 μm Whatman filter. The solution previously obtained is rapidly added to 3 ml of water at ambient temperature to precipitate the crystal, which is recovered by filtration and dried under vacuum. The product (crystalline form α) presents an IR spectrum, DSC curve and XRPD pattern as shown in FIGS. 1-3 respectively.

### Example 8

Venetoclax free base (100 mg) is dissolved in 2 ml of tetrahydrofuran under magnetic stirring for about an hour, then filtered through an 0.45 μm Whatman filter. The solution previously obtained is rapidly added to 3 ml of water at the temperature of 0-5° C. to precipitate the crystal, which is recovered by filtration and dried under vacuum. The product (crystalline form α) presents an IR spectrum, DSC curve and XRPD pattern as shown in FIGS. 1-3 respectively.

### Example 9

Venetoclax free base (100 mg) is dissolved in 2 ml of tetrahydrofuran under magnetic stirring for about an hour, then filtered through an 0.45 μm Whatman filter. The solution previously obtained is added drop by drop to 3 ml of water at ambient temperature to precipitate the crystal, which is recovered by filtration and dried under vacuum. The product (crystalline form α) presents an IR spectrum, DSC curve and XRPD pattern as shown in FIGS. 1-3 respectively.

### Example 10

Venetoclax free base (100 mg) is dissolved in 2 ml of tetrahydrofuran under magnetic stirring for about an hour, then filtered through an 0.45 μm Whatman filter. The solution previously obtained is added drop by drop to 3 ml of water at the temperature of 0-5° C. to precipitate the crystal, which is recovered by filtration and dried under vacuum. The product (crystalline form α) presents an IR spectrum, DSC curve and XRPD pattern as shown in FIGS. 1-3 respectively.

### Example 11

Venetoclax free base (500 mg) is suspended in 10 ml of isopropanol under magnetic stirring for about 24 hours at ambient temperature. The crystal is then filtered and dried under vacuum. The product (crystalline form β) presents an IR spectrum, DSC curve and XRPD pattern as shown in FIGS. 5-7 respectively.

### Example 12

Venetoclax free base (500 mg) is suspended in 10 ml of ethyl acetate under magnetic stirring for about 24 hours at ambient temperature. The crystal is then filtered and dried under vacuum. The product (crystalline form γ) presents an IR spectrum, DSC curve and XRPD pattern as shown in FIGS. 9-11 respectively.

### Example 13

Venetoclax free base (500 mg) is suspended in 10 ml of dioxane under magnetic stirring for about 24 hours at ambient temperature. The crystal is then filtered and dried under vacuum. The product (crystalline form δ) presents an IR spectrum, DSC curve and XRPD pattern as shown in FIGS. 13-15 respectively.

### Example 14

Venetoclax free base (500 mg) is suspended in 10 ml of tert-butyl methyl ether under magnetic stirring for about 24 hours at ambient temperature. The crystal is then filtered and dried under vacuum. The product (crystalline form s) presents an IR spectrum, DSC curve and XRPD pattern as shown in FIGS. 17-19 respectively.

### Example 15

Venetoclax free base (500 mg) is suspended in 10 ml of heptane under magnetic stirring for about 24 hours at ambient temperature. The crystal is then filtered and dried under vacuum. The product (crystalline form θ) presents an IR spectrum, DSC curve and XRPD pattern as shown in FIGS. 21-23 respectively.

1. The crystalline form α of venetoclax characterized by:
an IR spectrum which shows absorption peaks at 3385.4, 3326.3, 2917.4, 2846.2, 1661.1, 1605.8, 1523.6, 1410.8, 1343.7, 1236.8, 1168.2, 1141.6, 1089.9, 981.9, 904.9, 812.1, 763.6, 738.6, 662.2±1.5 cm⁻¹;
a DSC curve which shows an endothermic peak at 142±1° C.;
an XRPD pattern comprising the following peaks (2θ): 4.61, 5.14, 5.44, 7.22, 7.98, 8.80, 9.21, 10.27, 10.95, 12.27, 13.84, 14.36, 15.15, 16.35, 17.96, 18.77, 20.02, 21.62, 24.91, 25.86, 29.19±0.2°;
THF residue percentage within the range of 0.05-0.4 mols per mole of venetoclax.

2.-6. (canceled)

7. A process for the preparation of the crystalline form α of venetoclax of claim 1 which comprises dissolving venetoclax in tetrahydrofuran, and slowly removing the solvent by evaporation and optionally recrystallization from tetrahydrofuran.

8.-12. (canceled)
